(19) United States
(12) Patent Application Publication
(10) Pub. No.: US 2024/0180694 A1
(43) Pub. Date: Jun. 6, 2024

(54) CARDIAC VALVE WITH FLOATING OUTER SKIRT FOR IMPROVED SEALING

(71) Applicant: BOSTON SCIENTIFIC SCIMED, INC., Maple Grove, MN (US)

(72) Inventors: Declan Loughnane, Galway (IE); Joseph Murphy, Galway (IE); Tim O'Connor, Galway (IE)

(73) Assignee: BOSTON SCIENTIFIC SCIMED, INC., MAPLE GROVE, MN (US)

(21) Appl. No.: 18/528,035

(22) Filed: Dec. 4, 2023

Related U.S. Application Data
(60) Provisional application No. 63/430,119, filed on Dec. 5, 2022.

(51) Int. Cl. A61F 2/24

(52) U.S. Cl. CPC A61F 2/2409; A61F 2220/0075; A61F 2250/001; A61F 2250/0063; A61F 2250/0069

(57) ABSTRACT

An implantable medical device is adapted to be implanted at an implantation site such as a native cardiac valve annulus. The implantable medical device includes an expandable frame that is adapted to expand from a collapsed configuration for delivery to an expanded configuration for deployment, one or more valve cusps secured relative to the expandable frame, an inner skirt surrounding a portion of the expandable frame, and an outer skirt surrounding a portion of the expandable frame, the outer skirt attached to the expandable frame via one or more sutures that axially secure the outer skirt relative to the expandable frame yet allow the outer skirt to move radially relative to the expandable frame. The outer skirt may move radially outwards in response to fluid pressure, thereby improving fluid sealing relative to the native cardiac valve annulus.

### CROSS REFERENCE TO RELATED APPLICATIONS

This application claims the benefit of priority of U.S. Provisional Application No. 63/430,119 filed Dec. 5, 2022, the entire disclosure of which is hereby incorporated by reference.

### TECHNICAL FIELD

The disclosure relates generally to medical devices and more particularly to replacement cardiac valves.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### SUMMARY

This disclosure provides design, material, manufacturing method, and use alternatives for medical devices. An example may be found in an implantable medical device that is adapted to be implanted at an implantation site. The implantable medical device includes an expandable frame that is adapted to expand from a collapsed configuration for delivery to an expanded configuration for deployment, one or more valve cusps secured relative to the expandable frame, an inner skirt surrounding a portion of the expandable frame, and an outer skirt surrounding a portion of the expandable frame, the outer skirt attached to the expandable frame via one or more sutures that axially secure the outer skirt relative to the expandable frame yet allow the outer skirt to move radially relative to the expandable frame.

Alternatively or additionally, the expandable frame may include an upper crown portion, a lower crown portion, commissural posts extending proximally from upper crown portion, and stabilization arches extending proximally from commissural posts.

Alternatively or additionally, each of the one or more sutures may be secured relative to one of the commissural posts.

Alternatively or additionally, each of the one or more sutures may extend beyond the corresponding commissural posts and may loop around a base of the stabilization arches above the corresponding commissural posts.

Alternatively or additionally, each of the sutures may be enveloped within the valve cusps secured relative to each of the commissural posts.

Alternatively or additionally, a distal edge of the outer skirt may be sewn relative to the inner skirt and a proximal edge of the outer skirt may overlie at least a portion of the upper crown portion.

Alternatively or additionally, each of the one or more sutures may be adapted to allow the proximal edge of the outer skirt to billow outwards in response to fluid pressure on the outer skirt.

Alternatively or additionally, both ends of each of the one or more sutures may attach to the outer skirt at a single position.

Alternatively or additionally, for each of the one or more sutures, a first end of a suture may attach to the outer skirt at a first position and a second end of the suture may attach to the outer skirt at a second position circumferentially spaced from the first position.

Another example may be found in a replacement cardiac valve that is adapted to be implanted within a native cardiac valve annulus. The replacement cardiac valve includes an expandable frame that is adapted to expand from a collapsed configuration for delivery to an expanded configuration for deployment and that includes an upper crown portion, a lower crown portion, commissural posts extending proximally from upper crown portion, and stabilization arches extending proximally from commissural posts. The replacement cardiac valve includes one or more valve cusps secured relative to the expandable frame, an inner skirt surrounding a portion of the expandable frame, and an outer skirt surrounding a portion of the expandable frame, the outer skirt adapted to billow outwards in response to fluid pressure on the outer skirt, thereby causing the outer skirt to seal against the native valve annulus.

Alternatively or additionally, the outer skirt may be attached to the expandable frame via one or more sutures that axially secure the outer skirt relative to the expandable frame yet allow the outer skirt to move radially relative to the expandable frame.

Alternatively or additionally, a distal edge of the outer skirt may be sewn relative to the inner skirt, and a proximal edge of the outer skirt may overlie at least a portion of the upper crown portion.

Alternatively or additionally, the inner skirt may cover the lower crown portion.

Alternatively or additionally, both ends of each of the one or more sutures may attach to the outer skirt at a single position.

Alternatively or additionally, for each of the one or more sutures, a first end of a suture may attach to the outer skirt at a first position and a second end of the suture may attach to the outer skirt at a second position circumferentially spaced from the first position.

Alternatively or additionally, the replacement cardiac valve may include a replacement aortic valve.

Another example may be found in a replacement aortic valve that is adapted to be implanted within a native aortic valve annulus. The replacement cardiac valve includes an expandable frame that is adapted to expand from a collapsed configuration for delivery to an expanded configuration for deployment and that includes an upper crown portion, a lower crown portion, commissural posts extending proximally from upper crown portion, and stabilization arches extending proximally from commissural posts. The replacement aortic valve includes one or more valve cusps secured relative to the expandable frame, and a sealing skirt surrounding a portion of the expandable frame, the sealing skirt adapted to billow outwards in response to aortic pressure on the sealing skirt when the one or more valve cusps are closed, thereby causing the sealing skirt to seal against the aortic valve annulus.

Alternatively or additionally, the sealing skirt may be attached to the expandable frame via one or more sutures that axially secure the sealing skirt relative to the expandable frame yet allow the sealing skirt to move radially relative to the expandable frame.

Alternatively or additionally, both ends of each of the one or more sutures may attach to the sealing skirt at a single position.

Alternatively or additionally, for each of the one or more sutures, a first end of a suture may attach to the sealing skirt at a first position and a second end of the suture may attach to the sealing skirt at a second position circumferentially spaced from the first position.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a view of an illustrative replacement aortic valve expandable frame;

FIG. 2 is a view of an illustrative replacement aortic valve utilizing the illustrative replacement aortic valve expandable frame of FIG. 1;

FIG. 3 is a view of an illustrative replacement aortic valve utilizing the illustrative replacement aortic valve expandable frame of FIG. 1; and

FIG. 4 is a graph showing experimental results.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about”, whether or not explicitly indicated. The term “about” generally refers to a range of numbers that one of skill in the art would consider equivalent to the recited value (e.g., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

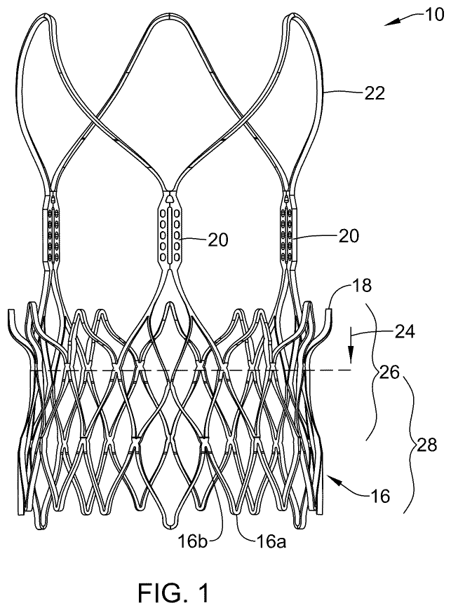

FIG. 1 is a side view of an illustrative replacement cardiac valve expandable frame 10. The replacement cardiac valve expandable frame 10 may be used as a starting point for building a replacement aortic valve, a replacement mitral valve, a replacement pulmonary valve or a replacement tricuspid valve, for example. In some cases, the resulting replacement cardiac valve may include biological tissue such as porcine or bovine pericardium and/or natural cardiac valve leaflets such as natural porcine cardiac valve leaflets that are secured relative to the replacement cardiac valve expandable frame 10. In some cases, the natural cardiac valve leaflets may be attached to a portion of natural cardiac wall tissue. The biological material may be fixed, for example, using glutaraldehyde.

The replacement cardiac valve expandable frame 10 may be compressible to a radially compressed, or collapsed, configuration for delivery using a delivery catheter, and may be expandable to an expanded configuration (as shown) during implantation. In some cases, the replacement cardiac valve expandable frame 10 may include a lower tubular or crown portion 16, an upper crown portion 18, a plurality of upstanding commissural posts 20, and a plurality of stabilization arches 22. In use, the lower portion 16 of the replacement cardiac valve expandable frame 10 may be adapted to be deployed after the other regions of the replacement cardiac valve expandable frame 10. For example, the arches 22, the supports 20 and the upper crown 18 may be deployed at least partly before the lower portion 16 (in that order, or in reverse order, or in a different order). At the very least, once the upper crown 18 has been at least partly deployed, the replacement cardiac valve expandable frame 10 may be urged and/or displaced in the direction of arrow 24 to seat the upper crown 18 against native leaflets at the implantation site. In this example, deploying the lower portion 16 last fixes the replacement cardiac valve expandable frame 10 in its final position.

The lower portion 16, and optionally a portion of the upper crown 18, may be formed by a lattice structure of the stent. The lattice structure may define cells or apertures, for example, generally diamond-shaped apertures. In some cases, the native leaflets may generally overlap a portion 26 of the replacement cardiac valve expandable frame 10. The native valve annulus may overlap a portion 28 of the expandable frame.

The replacement cardiac valve expandable frame 10 may optionally be of a self-expanding type that is compressible to the compressed configuration for loading into a delivery catheter for delivery to the site of implantation. In use, by removal of the constraining effect of a sheath holding the replacement cardiac valve expandable frame 10 in the compressed configuration, the replacement cardiac valve expandable frame 10 self-expands to or towards the operative configuration. A self-expanding stent may, for example, be of shape-memory material, for example, shape-memory metal alloy, for example, nitinol. Alternatively, the replacement cardiac valve expandable frame 10 may be configured to be expanded by application of a foreshortening force from the delivery catheter and/or by application of expanding force from the delivery catheter, such as by using an expansion balloon. These are just examples.

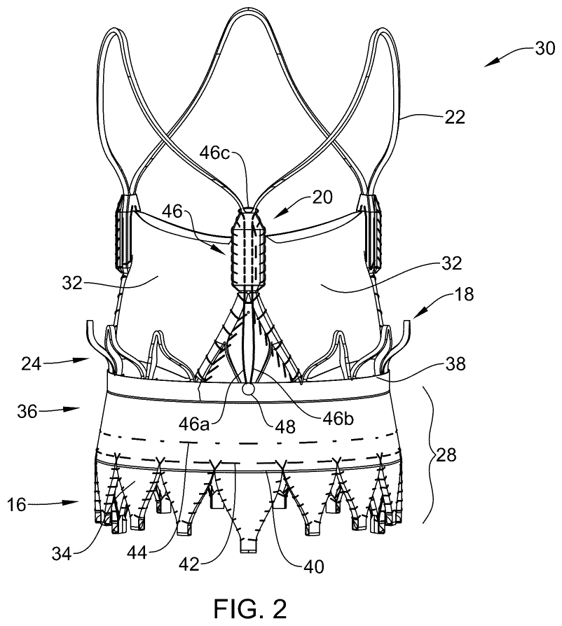

FIG. 2 is a side view of an illustrative replacement cardiac valve 30 that may be constructed using the replacement cardiac valve expandable frame 10 as a starting point. The illustrative replacement cardiac valve 30 includes several valve cusps 32. While a total of two valve cusps 32 are visible in this view, in some cases the replacement cardiac valve 30 may include any number of valve cusps 32, and in some cases may include a total of three valve cusps 32 that are equidistantly spaced about the replacement cardiac valve expandable frame 10. In some cases, for example, the valve cusps 32 may be formed of biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde. The valve cusps 32 may be secured in place relative to the replacement cardiac valve expandable frame 10 via adhesive or being sewn into place.

The replacement cardiac valve 30 includes an inner skirt 34 that envelopes the lower crown portion 16 and in some cases at least part of the upper crown portion 18 of the replacement cardiac valve expandable frame 10. The inner skirt 34 may be formed of biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde. The inner skirt 34 may be secured in place relative to the replacement cardiac valve expandable frame 10 via any of a number of ways including adhesive or being sewn into place.

The replacement cardiac valve 30 includes an outer skirt 36 that overlaps with the inner skirt 34. The outer skirt 36 may be considered as being a sealing skirt, as will be discussed. The outer skirt 36 may be considered as having a proximal region 38 and a distal region 40. In some cases, the distal region 40 of the outer skirt 36 may be sewn into place, such as via a line of sutures 42 and/or a line of sutures 44. The line of sutures 42 and/or the line of sutures 44 may secure the distal region 40 of the outer skirt 36 to the inner skirt 34. In some cases, the line of sutures 42 and/or the line of sutures 44 may secure the distal region 40 of the outer skirt 36 to the replacement cardiac valve expandable frame 10.

In some cases, the proximal region 38 of the outer skirt 36 may be attached to the replacement cardiac valve expandable frame 10 by virtue of one or more sutures 46. The one or more sutures 46 may be formed of any suitable thread or wire. As an example, the one or more sutures 46 may be PTFE (polytetrafluoroethylene) coated braided polyester (such as PET, or polyethylene terephthalate). While a single suture 46 is shown, it will be appreciated that there may be several sutures 46 distributed about the replacement cardiac valve 30. As an example, there may be a suture 46 positioned to interact with each of the commissure posts 20. Each suture 46 may be considered as having a first free end 46a and a second free end 46b. The suture 46 extends from the first free end 46a to a point 46c where the suture 46 loops over a portion of the stabilization arches 22, and back to the second free end 46b. While the suture 46 is shown as looping over a portion of the stabilization arches 22, in some cases each suture 46 may instead be secured relative to the commissural post 20, or even secured to the frame struts leading proximally towards the commissural posts 20.

As shown in FIG. 2, the first free end 46a and the second free end 46b are both secured to the proximal region 38 of the outer skirt 36 at a common point 48. The replacement cardiac valve 30 may be considered as a first version of a parachute seal, by virtue of how the sutures 46 are secured to the proximal region 38 of the outer skirt 36. Because the proximal region 38 of the outer skirt 36 is secured via the suture 46, rather than by tying knots, the proximal region 38 of the outer skirt 36 is free to expand or balloon in a radially outward direction as a result of fluid pressure against an interior of the outer skirt 36 when the valve cusps 32 are in a closed position. As the outer skirt 36 fills with blood and expands radially, the outer skirt 36 helps to seal against leaks between the outer skirt 36 and the native valve annulus.

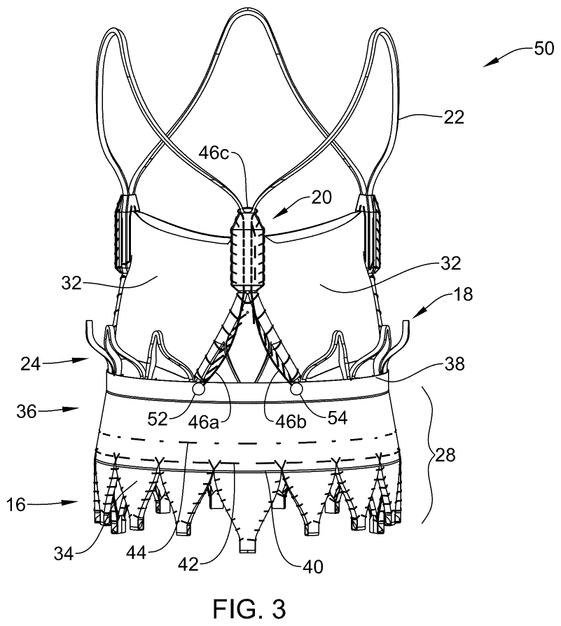

FIG. 3 is a side view of an illustrative replacement cardiac valve 50 that may be constructed using the replacement cardiac valve expandable frame 10 as a starting point. The illustrative replacement cardiac valve 50 includes several valve cusps 32. While a total of two valve cusps 32 are visible in this view, in some cases the replacement cardiac valve 50 may include any number of valve cusps 32, and in some cases may include a total of three valve cusps 32 that are equidistantly spaced about the replacement cardiac valve expandable frame 10. In some cases, for example, the valve cusps 32 may be formed of biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde. The valve cusps 32 may be secured in place relative to the replacement cardiac valve expandable frame 10 via any of a number of ways including adhesive or being sewn into place.

The replacement cardiac valve 50 includes an inner skirt 34 that envelopes the lower crown portion 16 and in some cases at least part of the upper crown portion 18 of the replacement cardiac valve expandable frame 10. The inner skirt 34 may be formed of biological tissue such as porcine or bovine pericardium and/or natural cardiac valve leaflets such as natural porcine cardiac valve leaflets. In some cases, the natural cardiac valve leaflets may be attached to a portion of natural cardiac wall tissue. The biological material may be fixed, for example, using glutaraldehyde. The inner skirt 34 may be secured in place relative to the replacement cardiac valve expandable frame 10 via adhesive or being sewn into place.

The replacement cardiac valve 50 includes an outer skirt 36 that overlaps with the inner skirt 34. The outer skirt 36 may be considered as being a sealing skirt, as will be discussed. The outer skirt 36 may be considered as having a proximal region 38 and a distal region 40. In some cases, the distal region 40 of the outer skirt 36 may be sewn into place, such a via a line of sutures 42 and/or a line of sutures 44. The line of sutures 42 and/or the line of sutures 44 may secure the distal region 40 of the outer skirt 36 to the inner skirt 34. In some cases, the line of sutures 42 and/or the line of sutures 44 may secure the distal region 40 of the outer skirt 36 to the replacement cardiac valve expandable frame 10.

In some cases, the proximal region 38 of the outer skirt 36 may be attached to the replacement cardiac valve expandable frame 10 by virtue of one or more sutures 46. While a single suture 46 is shown, it will be appreciated that there may be several sutures 46 distributed about the replacement cardiac valve 50. As an example, there may be a suture 46 positioned to interact with each of the commissure posts 20. Each suture 46 may be considered as having a first free end 46a and a second free end 46b. The suture 46 extends from the first free end 46a to a point 46c where the suture 46 loops over a portion of the stabilization arches 22, and back to the second free end 46b. While the suture 46 is shown as looping over a portion of the stabilization arches 22, in some cases each suture 46 may instead be secured relative to the commissural post 20, or even secured to the frame struts leading proximally towards the commissural posts 20.

As shown in FIG. 3, the first free end 46a is secured to the proximal region 38 of the outer skirt 36 at a first position 52 and the second free end 46b is secured to the proximal region 38 of the outer skirt 36 at a second position 54 that is circumferentially spaced from the first position 52. The replacement cardiac valve 50 may be considered as a second version of a parachute seal, by virtue of how the sutures 46 are secured to the proximal region 38 of the outer skirt 36. Because the proximal region 38 of the outer skirt 36 is secured via the suture 46, rather than by tying knots, the proximal region 38 of the outer skirt 36 is free to expand or balloon in a radially outward direction as a result of fluid pressure against an interior of the outer skirt 36 when the valve cusps 32 are in a closed position. As the outer skirt 36 fills with blood and expands radially, the outer skirt 36 helps to seal against leaks between the outer skirt 36 and the native valve annulus.

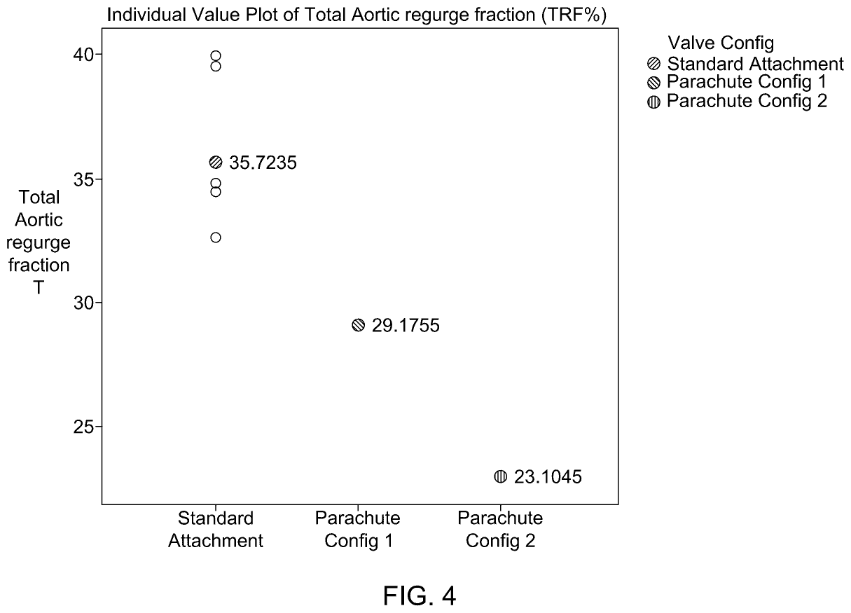

FIG. 4 is a graph providing experimental evidence showing improvements in scaling performance by virtue of securing the proximal region 38 of the outer skirt 36 to the replacement cardiac valve expandable frame 10 using the sutures 46 that allow radial movement of at least the proximal region 38 of the outer skirt 36. In the graph, the data point for the “standard attachment” references a replacement cardiac valve such as the replacement aortic valve 30 or the replacement aortic valve 50, but in which the proximal region 38 of the outer skirt 36 is secured in place by a series of knots that are circumferentially spaced about a periphery of the proximal region 38 of the outer skirt 36. It will be appreciated that the outer skirt 36 is not permitted to expand radially in the areas proximate each of the knots. In some cases, there may be a total of six knots.

The data point labeled “Parachute Config 1” corresponds to a valve such as the replacement aortic valve 30, in which the free ends 46a and 46b of each suture 46 are secured at a common point 48. The data point labeled “Parachute Config 2” corresponds to a valve such as the replacement aortic valve 50, in which the free ends 46a and 46b of each suture 46 are secured at spaced apart points 52 and 54. As can be seen, the data point labeled “Parachute Config 1” represents an 18.3 percent decrease in total aortic regurge fraction relative to the “standard attachment” data point. The data point labeled “Parachute Config 2” is better yet, representing a 35.3 percent decrease in total aortic regurge fraction relative to the “standard attachment” data point.

The materials that can be used for the devices described herein may include those commonly associated with medical devices. The devices described herein, or components thereof, may be made from a metal, metal alloy, polymer (some examples of which are disclosed below), a metal-polymer composite, ceramics, combinations thereof, and the like, or other suitable material. Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super-elastic nitinol in that the linear elastic and/or non-super-elastic nitinol does not display a substantial “superelastic plateau” or “flag region” in its stress/strain curve like super-elastic nitinol does. Instead, in the linear elastic and/or non-super-clastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-clastic nitinol may also be distinguishable from super-elastic nitinol in that linear elastic and/or non-super-elastic nitinol may accept up to about 2-5% strain while remaining substantially clastic (e.g., before plastically deforming) whereas super elastic nitinol may accept up to about 8% strain before plastically deforming. Both of these materials can be distinguished from other linear elastic materials such as stainless steel (that also can be distinguished based on its composition), which may accept only about 0.2 to 0.44 percent strain before plastically deforming.

In some embodiments, the linear clastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (C) to about 120° ° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-clastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, the devices described herein, or components thereof, may also be doped with, made of, or otherwise include a radiopaque material. Radiopaque materials are understood to be materials capable of producing a relatively bright image on a fluoroscopy screen or another imaging technique during a medical procedure. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of guidewire 10 to achieve the same result.

In some embodiments, a degree of Magnetic Resonance Imaging (MRI) compatibility is imparted into the devices described herein, or components thereof. For example, the devices described herein, or components thereof, may be made of a material that does not substantially distort the image and create substantial artifacts (e.g., gaps in the image). Certain ferromagnetic materials, for example, may not be suitable because they may create artifacts in an MRI image. The devices described herein, or components thereof, may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-NR and the like), nitinol, and the like, and others.

A sheath or covering (not shown) may be disposed over portions or all of the devices described herein in order to define a generally smooth outer surface. In other embodiments, however, such a sheath or covering may be absent. The sheath may be made from a polymer or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), MARLEX® high-density polyethylene, MARLEX® low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

In some embodiments, the exterior surface of the devices described herein may be sandblasted, beadblasted, sodium bicarbonate-blasted, electropolished, etc. In these as well as in some other embodiments, a coating, for example a lubricious, a hydrophilic, a protective, or other type of coating may be applied. Alternatively, a sheath may include a lubricious, hydrophilic, protective, or other type of coating. Hydrophobic coatings such as fluoropolymers provide a dry lubricity which improves guidewire handling and device exchanges. Lubricious coatings improve steerability and improve lesion crossing capability. Suitable lubricious polymers are well known in the art and may include silicone and the like, hydrophilic polymers such as high-density polyethylene (HDPE), polytetrafluoroethylene (PTFE), polyarylene oxides, polyvinylpyrrolidones, polyvinylalcohols, hydroxy alkyl cellulosics, algins, saccharides, caprolactones, and the like, and mixtures and combinations thereof. Hydrophilic polymers may be blended among themselves or with formulated amounts of water insoluble compounds (including some polymers) to yield coatings with suitable lubricity, bonding, and solubility. Some other examples of such coatings and materials and methods used to create such coatings can be found in U.S. Pat. Nos. 6,139,510 and 5,772,609, which are incorporated herein by reference.

Portions of the devices described herein may be formed, for example, by coating, extrusion, co-extrusion, interrupted layer co-extrusion (ILC), or fusing several segments end-to-end. The layer may have a uniform stiffness or a gradual reduction in stiffness from the proximal end to the distal end thereof. The gradual reduction in stiffness may be continuous as by ILC or may be stepped as by fusing together separate extruded tubular segments. The outer layer may be impregnated with a radiopaque filler material to facilitate radiographic visualization. Those skilled in the art will recognize that these materials can vary widely without deviating from the scope of the present disclosure.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. An implantable medical device adapted to be implanted at an implantation site, the implantable medical device comprising:
an expandable frame that is adapted to expand from a collapsed configuration for delivery to an expanded configuration for deployment;
one or more valve cusps secured relative to the expandable frame;
an inner skirt surrounding a portion of the expandable frame; and
an outer skirt surrounding a portion of the expandable frame, the outer skirt attached to the expandable frame via one or more sutures that axially secure the outer skirt relative to the expandable frame yet allow the outer skirt to move radially relative to the expandable frame.

2. The implantable medical device of claim 1, wherein the expandable frame comprises:
an upper crown portion;
a lower crown portion;
commissural posts extending proximally from upper crown portion; and
stabilization arches extending proximally from commissural posts.

3. The implantable medical device of claim 2, wherein each of the one or more sutures are secured relative to one of the commissural posts.

4. The implantable medical device of claim 2, wherein each of the one or more sutures extend beyond the corresponding commissural posts and loop around a base of the stabilization arches above the corresponding commissural posts.

5. The implantable medical device of claim 4, wherein each of the sutures are enveloped within the valve cusps secured relative to each of the commissural posts.

6. The implantable medical device of claim 2, wherein:
a distal edge of the outer skirt is sewn relative to the inner skirt; and
a proximal edge of the outer skirt overlies at least a portion of the upper crown portion.

7. The implantable medical device of claim 1, wherein each of the one or more sutures are adapted to allow the proximal edge of the outer skirt to billow outwards in response to fluid pressure on the outer skirt.

8. The implantable medical device of claim 1, wherein both ends of each of the one or more sutures attach to the outer skirt at a single position.

9. The implantable medical device of claim 1, wherein for each of the one or more sutures, a first end of a suture attaches to the outer skirt at a first position and a second end of the suture attaches to the outer skirt at a second position circumferentially spaced from the first position.

10. A replacement cardiac valve adapted to be implanted within a native cardiac valve annulus, the replacement cardiac valve comprising:
an expandable frame that is adapted to expand from a collapsed configuration for delivery to an expanded configuration for deployment, the expandable frame including
an upper crown portion;
a lower crown portion;
commissural posts extending proximally from upper crown portion; and
stabilization arches extending proximally from commissural posts;
one or more valve cusps secured relative to the expandable frame;
an inner skirt surrounding a portion of the expandable frame; and
an outer skirt surrounding a portion of the expandable frame, the outer skirt adapted to billow outwards in response to fluid pressure on the outer skirt, thereby causing the outer skirt to seal against the native valve annulus.

11. The replacement cardiac valve of claim 10, wherein the outer skirt is attached to the expandable frame via one or more sutures that axially secure the outer skirt relative to the expandable frame yet allow the outer skirt to move radially relative to the expandable frame.

12. The replacement cardiac valve of claim 10, wherein
a distal edge of the outer skirt is sewn relative to the inner skirt; and
a proximal edge of the outer skirt overlies at least a portion of the upper crown portion.

13. The replacement cardiac valve of claim 10, wherein the inner skirt covers the lower crown portion.

14. The replacement cardiac valve of claim 10, wherein both ends of each of the one or more sutures attach to the outer skirt at a single position.

15. The replacement cardiac valve of claim 10, wherein for each of the one or more sutures, a first end of a suture attaches to the outer skirt at a first position and a second end of the suture attaches to the outer skirt at a second position circumferentially spaced from the first position.

16. The replacement cardiac valve of claim 10, comprising a replacement aortic valve.

17. A replacement aortic valve adapted to be implanted within a native aortic valve annulus, the replacement cardiac valve comprising:
an expandable frame that is adapted to expand from a collapsed configuration for delivery to an expanded configuration for deployment, the expandable frame including
an upper crown portion;
a lower crown portion;
commissural posts extending proximally from upper crown portion; and
stabilization arches extending proximally from commissural posts;
one or more valve cusps secured relative to the expandable frame; and
a sealing skirt surrounding a portion of the expandable frame, the sealing skirt adapted to billow outwards in response to aortic pressure on the sealing skirt when the one or more valve cusps are closed, thereby causing the sealing skirt to seal against the aortic valve annulus.

18. The replacement aortic valve of claim 17, wherein the sealing skirt is attached to the expandable frame via one or more sutures that axially secure the sealing skirt relative to the expandable frame yet allow the sealing skirt to move radially relative to the expandable frame.

19. The replacement cardiac valve of claim 17, wherein both ends of each of the one or more sutures attach to the sealing skirt at a single position.

20. The replacement cardiac valve of claim 17, wherein for each of the one or more sutures, a first end of a suture attaches to the sealing skirt at a first position and a second end of the suture attaches to the sealing skirt at a second position circumferentially spaced from the first position.
